(19) United States
(12) Patent Application Publication
(10) Pub. No.: US 2025/0188034 A1
(43) Pub. Date: Jun. 12, 2025

(54) IONIC COMPOUND, ABSORBENT AND ABSORPTION DEVICE

(71) Applicant: Industrial Technology Research Institute, Hsinchu (TW)

(72) Inventors: Wei-Chih LEE, Hsinchu City (TW); Ai-Yu LIOU, Keelung City (TW); Chih-Hao CHEN, Taipei City (TW); Jyi-Ching PERNG, Hukou Township (TW); Jiun-Jen CHEN, New Taipei City (TW)

(73) Assignee: Industrial Technology Research Institute, Hsinchu (TW)

(21) Appl. No.: 18/646,452

(22) Filed: Apr. 25, 2024

(30) Foreign Application Priority Data
Dec. 8, 2023 (TW) 112147883

(51) Int. Cl. C07D 213/74; B01D 53/14; B01D 53/18; B01D 53/26; B01D 53/28; C07F 9/11

(52) U.S. Cl. CPC C07D 213/74; B01D 53/1493; B01D 53/18; B01D 53/263; B01D 53/28; C07F 9/11; B01D 2252/30; B01D 2257/80; B01D 2258/06

(57) ABSTRACT

An ionic compound, an absorbent and an absorption device are provided. The ionic compound has a structure represented by Formula (I)

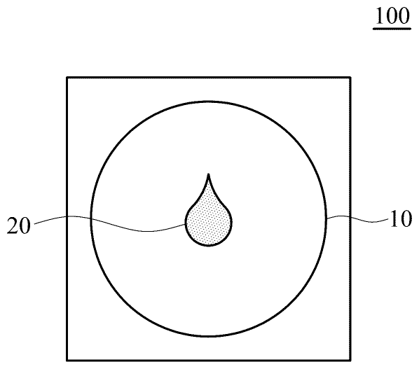

AB Formula (I), wherein A is

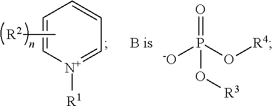

R¹ is C1-C6 alkyl group; R² is independently hydroxy group, amino group, C1-C6 alkyl group, C1-C6 alkoxy group, C1-C6 alkylamino group, C2-C7 alkoxycarbonyl group, C2-C7 alkanoyloxy group, carbamoyl group, C2-C13 alkylcarbamoyl group, or C2-C7 alkanoylamino group; n is 1, 2, 3, 4 or 5; and R³ and R⁴ are independently H, or C1-C6 alkyl group.

### CROSS REFERENCE TO RELATED APPLICATIONS

The application claims priority of Taiwan Patent Application No. 112147883, filed on Dec. 8, 2023, the entirety of which is incorporated by reference herein.

### BACKGROUND OF THE INVENTION

### Field of the Invention

The disclosure relates to an ionic compound, an absorbent, and an absorption device thereof.

### Description of the Related Art

Liquid absorbents have several advantages. They have high water-vapor absorption, low regeneration temperature, and low energy consumption. They are easy to transport, and waste heat long-distance utilization. As a result, they are widely used in dehumidification systems. Liquid absorbent dehumidification systems are key pieces of equipment for reducing carbon emissions and may aid in raising corporate ESG scores in the future.

Liquid absorbents are liquid materials that can directly absorb water vapor from the air to achieve a dehumidification effect. The driving force is the difference between the water vapor pressure in the air and the saturation vapor pressure of the liquid absorbent surface. Conventional liquid absorbents include aqueous solutions of chlorinated lithium, lithium bromide, lithium chloride, calcium chloride, and magnesium chloride. However, these salt solutions have strong corrosive properties on metals, which can seriously affect the reliability and service life of the dehumidification system. Although titanium can be used as a heat exchanger to avoid corrosion, this significantly increases the cost of building the dehumidification system. In addition, conventional salt solutions tend to crystallize and precipitate when saturated, leading to poor dehumidification performance and serious maintenance issues in the system, such as blockages of the circulation pump.

Both academia and industry have proposed using ionic compounds as liquid absorbents. However, conventional ionic compounds used as liquid absorbents still have properties that make them corrosive to metals, and their water vapor absorption and desorption capabilities still require improvement.

### SUMMARY

According to embodiments of the disclosure, the disclosure provides an ionic compound. the ionic compound having a structure represented by Formula (I), wherein A is

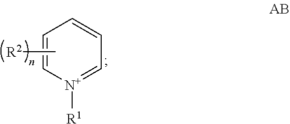

### B is

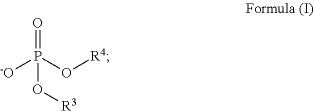

R¹ is C1-C6 alkyl group; R² is independently hydroxy group, amino group, C1-C6 alkyl group, C1-C6 alkoxy group, C1-C12 alkylamino group, C2-C7 alkoxycarbonyl group, C2-C7 alkanoyloxy group, carbamoyl group, C2-C13 alkylcarbamoyl group, or C2-C7 alkanoylamino group; n is 1, 2, 3, 4, or 5; and R³ and R⁴ are independently H, or C1-C6 alkyl group.

According to embodiments of the disclosure, the disclosure also provides an absorbent. The absorbent includes the ionic compound of the disclosure.

According to embodiments of the disclosure, the disclosure also provides a absorption device. The absorption device includes a chamber, and the absorbent of the disclosure, wherein the absorbent is disposed in the chamber.

### BRIEF DESCRIPTION OF THE DRAWINGS

The present invention can be more fully understood by reading the subsequent detailed description and examples with references made to the accompanying drawings, wherein:

FIGURE is a schematic view of the absorption device according to embodiments of the disclosure.

### DETAILED DESCRIPTION OF DISCLOSED EMBODIMENTS

The ionic compound, absorbent, and absorption device of the disclosure are described in detail in the following description. In the following detailed description, for purposes of explanation, numerous specific details and embodiments are set forth in order to provide a thorough understanding of the present disclosure. The specific elements and configurations described in the following detailed description are set forth in order to clearly describe the present disclosure. It will be apparent, however, that the exemplary embodiments set forth herein are used merely for the purpose of illustration, and the inventive concept may be embodied in various forms without being limited to those exemplary embodiments. In addition, the drawings of different embodiments may use like and/or corresponding numerals to denote like and/or corresponding elements in order to clearly describe the present disclosure. However, the use of like and/or corresponding numerals in the drawings of different embodiments does not suggest any correlation between different embodiments. As used herein, the term “about” in quantitative terms refers to plus or minus an amount that is general and reasonable to persons skilled in the art.

The disclosure provides an ionic compound, absorbent, and absorption device employing the same. According to embodiments of the disclosure, the ionic compound consists of a cationic moiety A and an anionic moiety B, wherein the cationic moiety A may be a quaternary ammonium cationic moiety derived from pyridine, and the anionic moiety B may be an anionic moiety derived from alkyl phosphate. Due to the combination of the specific cationic moiety A and the specific anionic moiety B, the liquid absorbent employing the ionic compound of the disclosure exhibits higher water-vapor absorption capacity and lower desorption temperature (able to desorb at 60° C. or lower), thereby increasing the dehumidification efficiency of the absorption device that utilizes the absorbent. In addition, the liquid absorbent employing the ionic compound of the disclosure has various advantages such as odorless, antibacterial, low corrosiveness, fluidity, and excellent room temperature stability, which can be widely applied to various dehumidification devices.

The ionic compound of the disclosure can be regenerated at low temperatures and exhibits high solubility in water. In comparison to conventional solid adsorbents, the ionic compound of the disclosure is not constrained by the distance from waste heat sources to end-users. This allows for the efficient utilization of industrial waste heat without the need for insulation during transportation, thereby completely overcoming previous limitations related to the distance of waste heat sources. The ionic compound of the disclosure possesses characteristics for the removal of volatile Organic compounds (VOCs) and has antimicrobial properties to maintain the air quality of incoming air, making it suitable for applications in constant temperature and humidity air conditioning systems in various fields such as residential, hospitals, biotechnology processes, warehouses, drying processes, special storage, and other related areas.

According to embodiments of the disclosure, the disclosure provides an ionic compound, such as an ionic liquid. the ionic compound having a structure represented by Formula (I), wherein A is

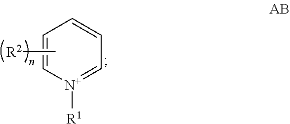

### B

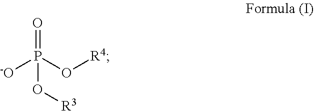

R¹ is C1-C6 alkyl group; R² is independently hydroxy group, amino group, C1-C6 alkyl group, C1-C6 alkoxy group, C1-C12 alkylamino group, C2-C7 alkoxycarbonyl group, C2-C7 alkanoyloxy group, carbamoyl group, C2-C13 alkylcarbamoyl group, or C2-C7 alkanoylamino group; n is 1, 2, 3, 4, or 5; and R³ and R⁴ are independently H, or C1-C6 alkyl group.

According to embodiments of the disclosure, alkyl group may be linear or branched alkyl group. According to embodiments of the disclosure, C1-C6 alkyl group may be methyl, ethyl, propyl, butyl, pentyl, hexyl, or an isomer thereof. For example, C1-C6 alkyl group may be methyl, ethyl, n-propyl, isopropyl, n-butyl, sec-butyl, iso-butyl, tert-butyl, n-pentyl, iso-pentyl, sec-pentyl, neopentyl, n-hexyl, iso-hexyl, sec-hexyl, or tert-hexyl.

According to embodiments of the disclosure, C1-C6 alkoxy group may be linear or branched alkoxy group. For example, C1-C6 alkoxy may be methoxy, ethoxy, propoxy, butoxy, pentoxy, hexoxy, or an isomer thereof.

According to embodiments of the disclosure, C1-C12 alkylamino group may be linear or branched alkylamino group. For example, C1-C12 alkylamino group may be —NH—CH₃, —N(CH₃)₂, —N(CH₂CH₃)₂, —N(CH₂CH₂CH₃)₂, or —N(CH₂CH₂ CH₂CH₃)₂.

According to embodiments of the disclosure, C2-C7 alkoxycarbonyl group may be linear or branched alkoxycarbonyl group. For example, C2-C7 alkoxycarbonyl group may be methoxycarbonyl, ethoxycarbonyl, or propoxycarbonyl.

According to embodiments of the disclosure, C2-C7 alkanoyloxy group may be linear or branched alkanoyloxy group. For example, C2-C7 alkanoyloxy group may be acetyloxy, propionyloxy, butyryloxy, pentylcarbonyloxy, hexylcarbonyloxy, or an isomer thereof.

According to embodiments of the disclosure, carbamoyl group may be

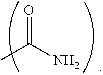

According to embodiments of the disclosure, C2-C13 alkylcarbamoyl group denotes a carbamoyl group in which at least one hydrogen bonded with nitrogen has been replaced with C1-C6 alkyl group. For example, C2-C13 alkylcarbamoyl group may be N-methylcarbamoyl, N,N-dimethylcarbamoyl, N,N-diethylcarbamoyl, or N,N-dipropylcarbamoyl.

According to embodiments of the disclosure, C2-C7 carbamoyl group is

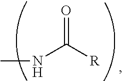

wherein R is C1-C6 alkyl group.

According to embodiments of the disclosure, when A is

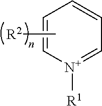

and n is 2, 3, 4, or 5, R² may be the same or different.

According to embodiments of the disclosure, the ionic compound may consists of a cationic moiety A and an anionic moiety B. According to embodiments of the disclosure, the cationic moiety A may be a monovalent quaternary ammonium cationic moiety derived from a substituted pyridine. According to embodiments of the disclosure, the substituted pyridine denotes that at least one of the hydrogen bonded with the carbon of pyridine may be optionally replaced with hydroxy group, amino group, C1-C6 alkyl group, C1-C6 alkoxy group, C1-C12 alkylamino group, C2-C7 alkoxycarbonyl group, C2-C7 alkanoyloxy group, carbamoyl group, C2-C13 alkylcarbamoyl group, or C2-C7 alkanoylamino group.

According to embodiments of the disclosure, the cationic moiety A may be

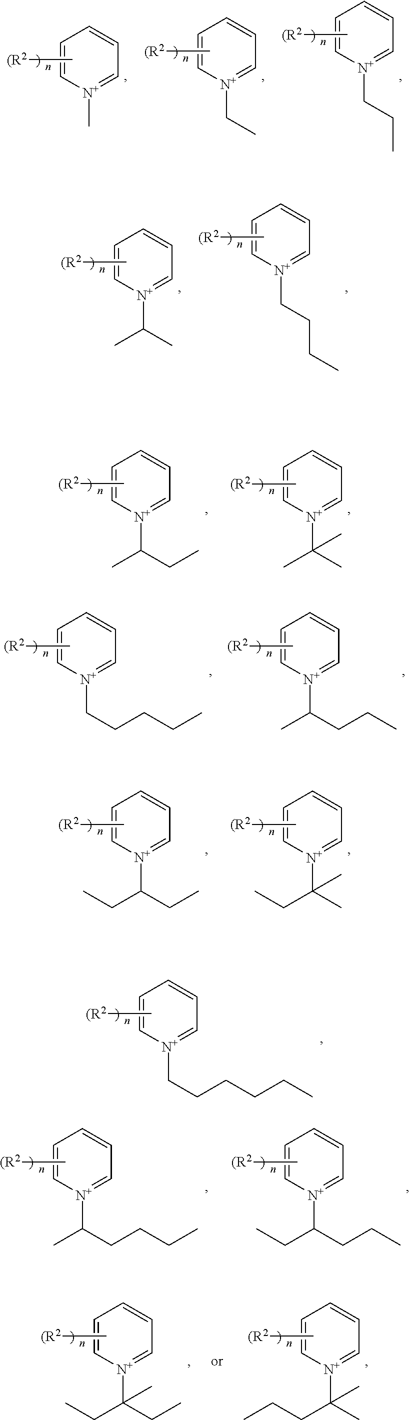

wherein R² is independently hydroxy group, amino group, C1-C6 alkyl group, C1-C6 alkoxy group, C1-C6 alkylamino group, C2-C7 alkoxycarbonyl group, C2-C7 alkanoyloxy group, C2-C13 alkylcarbamoyl group, or C2-C7 alkanoylamino group; n is 1, 2, 3, 4, or 5; and R³ and R⁴ are independently H, or C1-C6 alkyl group.

According to some embodiments of the disclosure, A may be

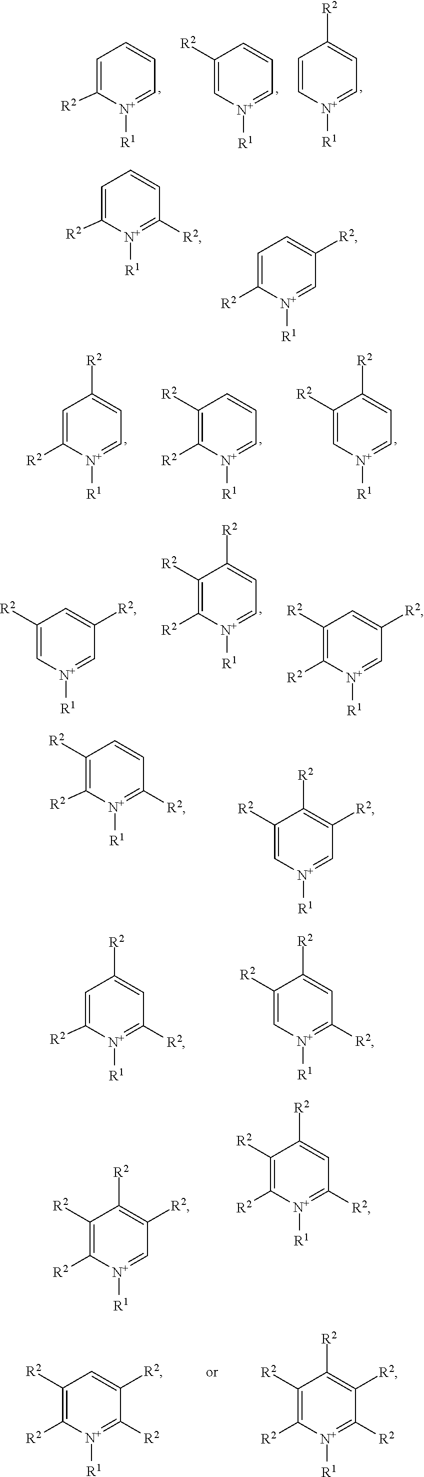

wherein R¹ is C1-C6 alkyl group; R² is independently hydroxy group, amino group, C1-C6 alkyl group, C1-C6 alkoxy group, C1-C6 alkylamino group, C2-C7 alkoxycarbonyl group, C2-C7 alkanoyloxy group, C2-C13 alkylcarbamoyl group, or C2-C7 alkanoylamino group.

According to some embodiments of the disclosure, A may be

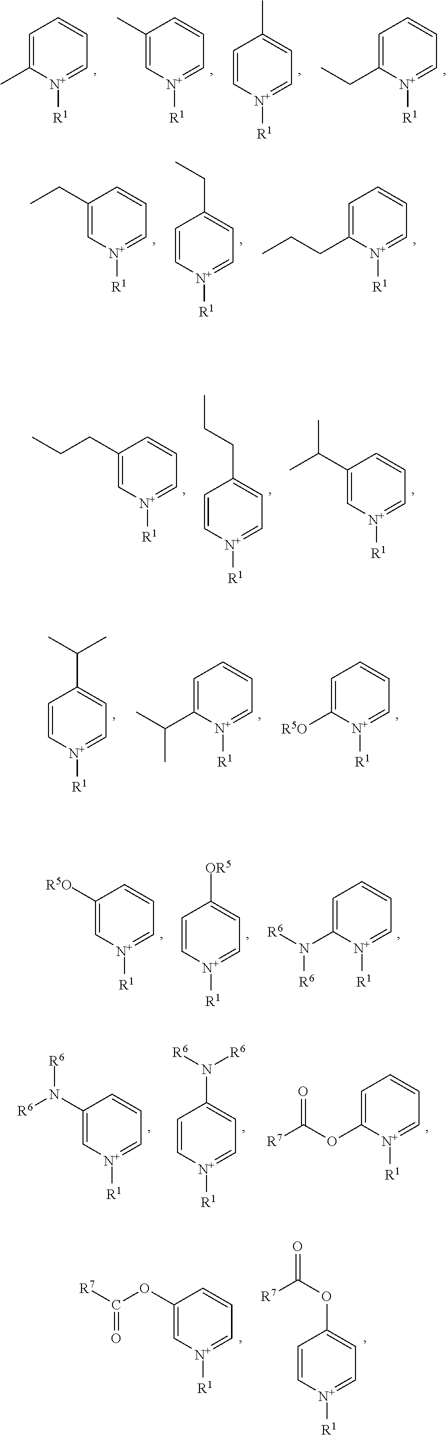

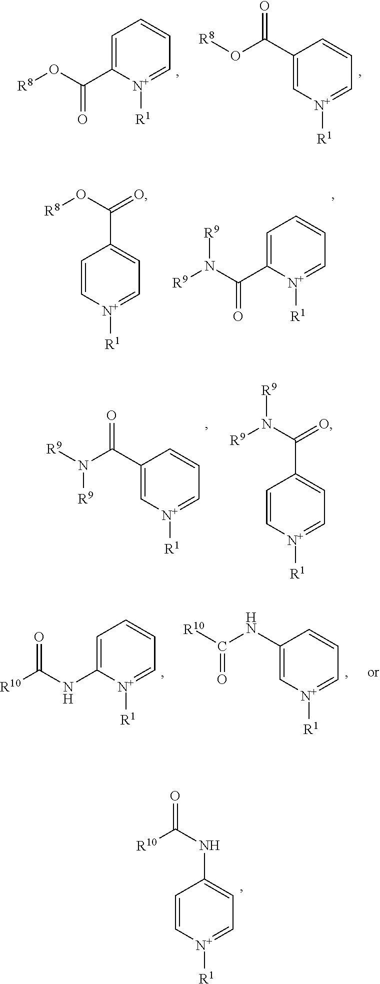

wherein R¹, R⁷, R⁸, and R¹⁰ are independently C1-C6 alkyl group; and R⁵, R⁶ and R⁹ are independently H, or C1-C6 alkyl group.

According to embodiments of the disclosure, the anionic moiety B may be an anionic moiety derived from alkyl phosphate,

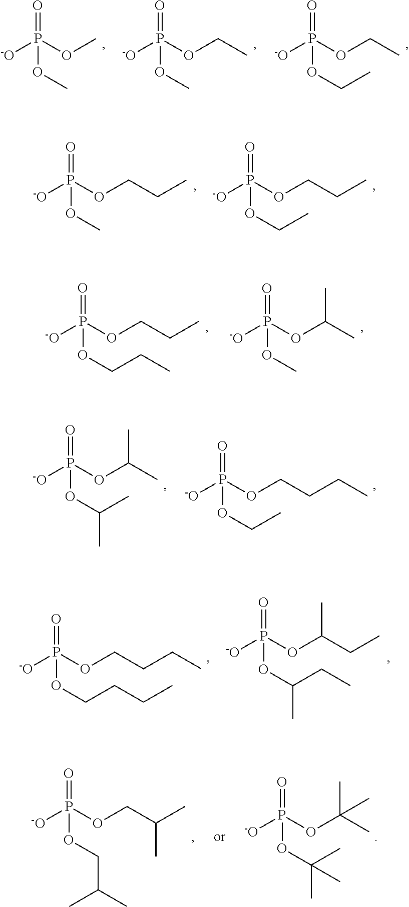

According to embodiments of the disclosure, the method for preparing the ionic compound having the monovalent quaternary ammonium cationic moiety may include following steps. First, a substituted pyridine and an anionic precursor is mixed, obtaining a mixture. According to embodiments of the disclosure, the anionic precursor may be alkyl phosphate (such as

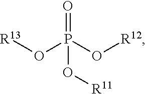

wherein R¹¹ is H, C1-C6 alkyl group, and R^{12,} and R¹³ may be independently C1-C6 alkyl group). According to embodiments of the disclosure, the molar ratio of the substituted pyridine to the anionic precursor may be 1:1 to 1:1.2, in order to form an ionic compound having the monovalent quaternary ammonium cationic moiety. Next, the mixture is subjected to a heating process, obtaining the ionic compound of the disclosure. According to embodiments of the disclosure, the heating process may have a temperature of about 50° C. to 150° C., such as 60° C., 70° C., 80° C., 90° C., 100° C., 110° C., 120° C., 130° C., or 140° C. According to embodiments of the disclosure, the heating process may have a time period of 1 hour to 24 hours, such as 2 hours, 4 hours, 6 hours, 8 hours, 10 hours, 12 hours, 16 hours, or 20 hours.

According to embodiments of the disclosure, before subjecting the mixture to a heating process, the mixture and a solvent were mixed, obtaining a solution. According to embodiments of the disclosure, the solvent may be toluene, methanol, ethanol, propanol, butanol, ethyl acetate, anisole, butyl acetate, or a combination thereof. According to embodiments of the disclosure, the solution has a solid content of about 5wt % to 95wt %, such as 10wt %, 20wt%, 30wt %, 40wt%, 50wt %, 60wt%, 70wt %, 80wt%, or 90wt %. Herein, the solid content means a weight percentage of the ingredients of the solution except the solvent, based on the total weight of the solution.

According to embodiments of the disclosure, the disclosure also provides an absorbent. According to embodiments of the disclosure, the absorbent can be utilized to absorb liquids (such as water) or gases (such as water vapor, ammonia, carbon dioxide, etc.).

According to embodiments of the disclosure, the absorbent may consist of the ionic compound of the disclosure. According to some embodiments of the disclosure, the absorbent of the disclosure includes the ionic compound of the disclosure and a solvent, in order to uniformly disperse or dissolve the ionic compound of the disclosure in the solvent. According to embodiments of the disclosure, the weight ratio of the ionic compound to the solvent may be about 1:9 to 9:1, such as 2:8, 3:7, 4:6, 5:5, 6:4, 7:3, or 8:2. According to embodiments of the disclosure, the solvent may be water, methanol, ethanol, propanol, butanol, ammonia, or a combination thereof.

According to embodiments of the disclosure, the absorbent of the disclosure includes ionic compound and water, wherein the weight ratio of ionic compound to water is 9:1 to 4:1. Herein, the absorbent may have a viscosity of 50 cP to 120 cP at 15° C., such as 60 cP, 80 cP, 100 cP, 110 cP.

According to embodiments of the disclosure, at the temperature range between 5° C. and 90° C., the state of the absorbent is liquid, i.e. the absorbent has a viscosity in a range of 10 cP to 500 cP.

According to embodiments of the disclosure, the absorbent of the disclosure has a relatively large vapor pressure difference at a specific temperature range (such as a range between 15° C. to 50° C. or a range between 15° C. to 60° C.), the absorbent exhibits high dehumidification efficiency and high desorption capability at low temperature.

According to embodiments of the disclosure, the disclosure also provides an absorption device. As shown in FIGURE, the absorption device 100 can include a chamber 10, and the absorbent of the disclosure 20, wherein the absorbent 20 is disposed in the chamber. According to embodiments of the disclosure, the chamber may be a absorb chamber, and the liquid or gas will be absorbed by the absorbent in the absorb chamber, achieving the purpose of reducing the liquid or gas content. Due to the extremely low metal corrosiveness of the absorbent of the disclosure, the installation cost of the absorption device could be reduced. Moreover, the absorbent of the disclosure is fluid at a temperature range of 5° C. to 90° C., which makes it suitable for applications in absorption devices that adopt spray or liquid flow processes.

Below, exemplary embodiments will be described in detail with reference to the accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

### EXAMPLE

### Preparation of Ionic Compound (1)

### Example 1

4-dimethylaminopyridine (DMAP) (0.60 mol) was added into a reaction bottle. Next, trimethyl phosphate (0.72 mol) was added into the reaction bottle dropwisely. After heating the reaction bottle at 150° C. for 30 minutes, the reaction bottle was heated at 100° C. for 8 hours. After cooling to room temperature, the result was dissolved in water (40 mL) and then extracted three times using diethyl ether (60 mL). Next, the organic volatiles and water were removed with a rotary concentrator. After baking the result in a vacuum oven at 90° C. for 8 hours, Ionic compound (1) was obtained (with a structure of

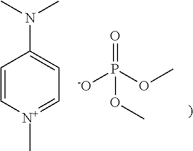

(white solid with a yield of 99%). The synthesis pathway of the above reaction was as follows:

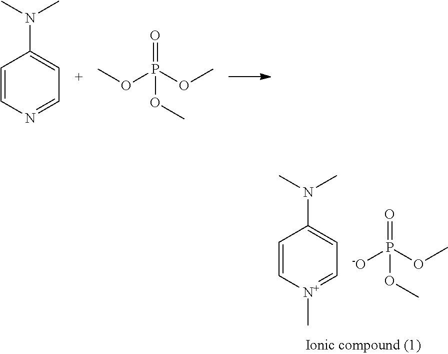

The measurement results of nuclear magnetic resonance spectrometry of Ionic compound (1) are shown below. ¹H NMR (400 MHz, ppm, CDCl₃): δ 8.53 (d, J=6.0 Hz, 2H), 6.96 (d, J=6.0 Hz, 2H), 4.11 (s, 3H), 3.60 (s, 3H), 3.58 (s, 3H), 3.21 (s, 6H).

### Example 2

4-dimethylaminopyridine (DMAP) (0.20 mol) was added into a reaction bottle. Next, triethyl phosphate (0.24 mol) was added into the reaction bottle dropwisely. After heating the reaction bottle at 150° C. for 30 minutes, the reaction bottle was heated at 100° C. for 8 hours. After cooling to room temperature, the result was dissolved in water (40 mL) and then extracted three times using diethyl ether (60 mL). Next, the organic volatiles and water were removed with a rotary concentrator. After baking the result in a vacuum oven at 90° C. for 8 hours, Ionic compound (2) was obtained (with a structure of

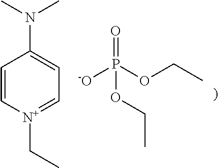

(white solid with a yield of 90%). The synthesis pathway of the above reaction was as follows:

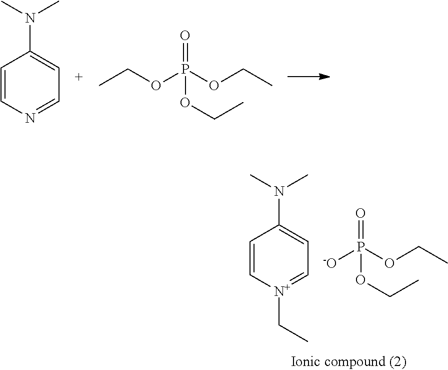

The measurement results of nuclear magnetic resonance spectrometry of Ionic compound (2) are shown below. ¹H NMR (400 MHz, ppm, CDCl₃): δ 8.49 (d, J=6.4 Hz, 2H), 7.03 (d, J=6.0 Hz, 2H), 4.37 (q, J=4.5 Hz, 2H), 3.95 (q, J=5.6 Hz, 4H), 3.23 (s, 6H), 1.53 (t, J=4.5 Hz, 3H), 1.24 (t, J=5.6 Hz, 6H).

### Example 3

2-methylpyridine (0.20 mol) was added into a reaction bottle. Next, trimethyl phosphate (0.24 mol) was added into the reaction bottle dropwisely. After heating the reaction bottle at 150° C. for 30 minutes, the reaction bottle was heated at 100° C. for 8 hours. After cooling to room temperature, the result was dissolved in water (40 mL) and then extracted three times using diethyl ether (60 mL). Next, the organic volatiles and water were removed with a rotary concentrator. After baking the result in a vacuum oven at 90° C. for 8 hours, Ionic compound (3) was obtained (with a structure of

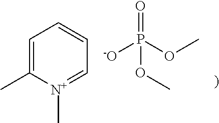

(brown solid with a yield of 95%). The synthesis pathway of the above reaction was as follows:

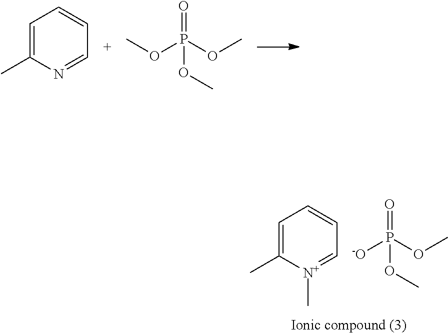

The measurement results of nuclear magnetic resonance spectrometry of Ionic compound (3) are shown below. ¹H NMR (400 MHz, ppm, CD₃OD): δ 8.87 (d, J=4.8 Hz, 1H), 8.46 (d, J=6.0 Hz, 1H), 8.01 (d, J=6.4 Hz, 1H), 7.91 (d, J=5.6 Hz, 1H), 4.31 (s, 3H), 3.59 (s, 3H), 3.56 (s, 3H), 2.86 (s, 3H).

### Example 4

3-methylpyridine (0.20 mol) was added into a reaction bottle. Next, trimethyl phosphate (0.24 mol) was added into the reaction bottle dropwisely. After heating the reaction bottle at 150° C. for 30 minutes, the reaction bottle was heated at 100° C. for 8 hours. After cooling to room temperature, the result was dissolved in water (40 mL) and then extracted three times using diethyl ether (60 mL). Next, the organic volatiles and water were removed with a rotary concentrator. After baking the result in a vacuum oven at 90° C. for 8 hours, Ionic compound (4) was obtained (with a structure of

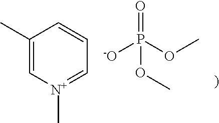

(brown liquid with a yield of 99%). The synthesis pathway of the above reaction was as follows:

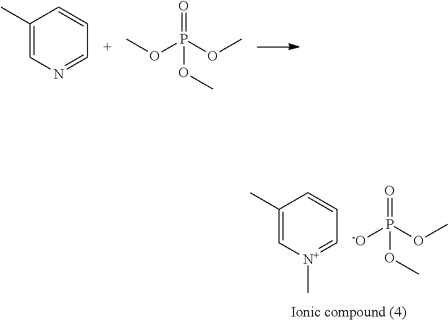

The measurement results of nuclear magnetic resonance spectrometry of Ionic compound (4) are shown below. ¹H NMR (400 MHz, ppm, CD₃OD): δ 8.81 (s, 1H), 8.74 (d, J=4.8 Hz, 1H), 8.43 (d, J=6.0 Hz, 1H), 7.99 (s, 1H), 4.39 (s, 3H), 3.59 (s, 3H), 3.56 (s, 3H), 2.59 (s, 3H).

### Example 5

4-methylpyridine (0.20 mol) was added into a reaction bottle. Next, trimethyl phosphate (0.24 mol) was added into the reaction bottle dropwisely. After heating the reaction bottle at 150° C. for 30 minutes, the reaction bottle was heated at 100° C. for 8 hours. After cooling to room temperature, the result was dissolved in water (40 mL) and then extracted three times using diethyl ether (60 mL). Next, the organic volatiles and water were removed with a rotary concentrator. After baking the result in a vacuum oven at 90° C. for 8 hours, Ionic compound (5) was obtained (with a structure of

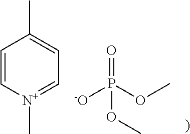

(brown solid with a yield of 99%). The synthesis pathway of the above reaction was as follows:

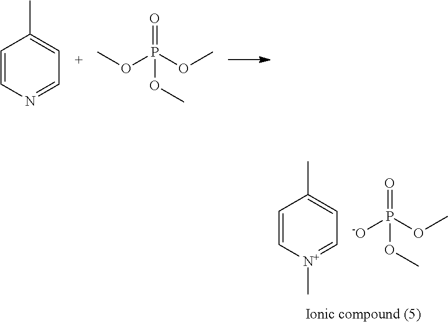

The measurement results of nuclear magnetic resonance spectrometry of Ionic compound (5) are shown below. ¹H NMR (400 MHz, ppm, CD₃OD): δ 8.73 (d, J=4.8 Hz, 2H), 7.93 (d, J=4.0 Hz, 2H), 4.35 (s, 3H), 3.59 (s, 3H), 3.57 (s, 3H), 2.69 (s, 3H).

### Comparative Example 1

Triethyl phosphate (0.30 mol) was added into a reaction bottle. Next, 1-methylimidazole (0.30 mol) was added into the reaction bottle dropwisely. After heating at 140° C. for 12 hours, the reaction bottle was cooled to room temperature, the result was dissolved in water (80 mL). After extracting three times using diethyl ether (50 mL), water phase was collected and an activated carbon (1 g) was added. After heating at 50° C. for 4 hours, the result was filtered. Next, the filtrate was concentrated, dehydrated and subjected to a freeze drying process, obtaining Ionic compound (6) (with a structure of

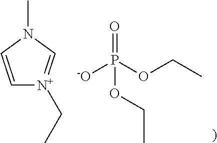

(yellow liquid, with a yield of 86%). The synthesis pathway of the above reaction was as follows:

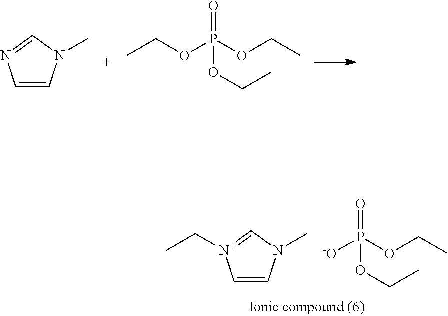

The measurement results of nuclear magnetic resonance spectrometry of Ionic compound (6) are shown below. ¹H NMR (400 MHz, ppm, CD₃OD): δ 8.95 (s, 1H), 7.66 (s, 1H), 7.59 (s, 1H), 4.27 (q, J=6.0 Hz, 2H), 3.94 (s, 3H), 3.91 (quint, J=5.6 Hz, 4H), 1.55 (t, J=6.0 Hz, 3H), 2.26 (t, J=5.6 Hz, 6H).

### Comparative Example 2

2-(N,N-dimethylamino)ethanol (DMAE) (0.1 mol) was added into a reaction bottle. Next, trimethyl phosphate (0.1 mol) was dropwisely added into the reaction bottle. After heating at 55° C. for 24 hours, the reaction bottle was cooled to room temperature, obtaining a white solid. The solid was dissolved in water (50 mL). After extracting three times using diethyl ether (30 mL), water phase was collected. After dehydrating with a rotary concentrator, the result was disposed in vacuum oven at 60° C. for 12 hours, obtaining Ionic compound (7) (with a structure of

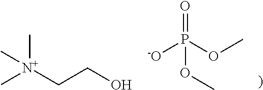

(light yellow liquid with a yield of 93%). The synthesis pathway of the above reaction was as follows:

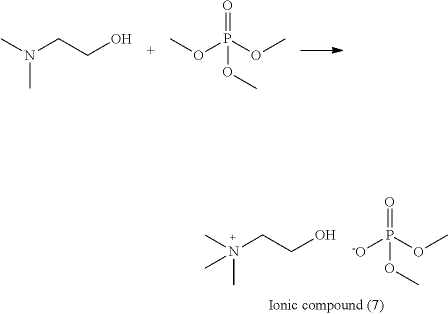

The measurement results of nuclear magnetic resonance spectrometry of Ionic compound (7) are shown below. ¹H NMR (400 MHz, ppm, CD₃OD): δ 3.99-3.96 (m, 2H), 3.55 (s, 3H), 3.53 (s, 3H), 3.48-3.46 (m, 2H), 3.20 (s, 9H).

### Comparative Example 3

Pyridine (0.21 mol) was added into a reaction bottle. Next, trimethyl phosphate (0.26 mol) was added into the reaction bottle dropwisely. After heating the reaction bottle at 150° C. for 30 minutes, the reaction bottle was heated at 100° C. for 8 hours. After cooling to room temperature, the result was dissolved in water (25 mL) and then extracted three times using diethyl ether (25 mL). Next, the organic volatiles and water were removed with a rotary concentrator. After baking the result in a vacuum oven at 90° C. for 8 hours, Ionic compound (8) was obtained (with a structure of

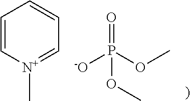

(brown liquid with a yield of 92%). The synthesis pathway of the above reaction was as follows:

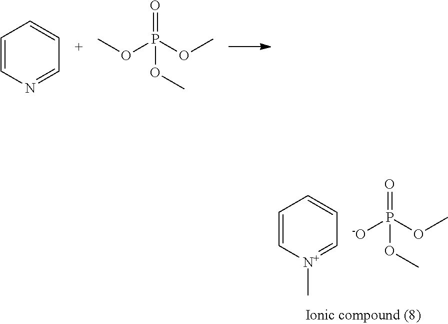

The measurement results of nuclear magnetic resonance spectrometry of Ionic compound (8) are shown below. ¹H NMR (400 MHz, ppm, CD₃OD): δ 9.45 (d, J=4.8 Hz, 2H), 8.39 (t, J=4.8 Hz, 1H), 8.02 (t, J=4.8 Hz, 2H), 4.59 (s, 3H), 3.50 (s, 3H), 3.48 (s, 3H).

### Comparative Example 4

4-dimethylaminopyridine (DMAP) (0.60 mol) was added into a reaction bottle. Next, diethyl sulfate (0.72 mol) was added into the reaction bottle dropwisely. After heating the reaction bottle at 150° C. for 30 minutes, the reaction bottle was heated at 100° C. for 8 hours. After cooling to room temperature, the result was dissolved in water (40 mL) and then extracted three times using ethyl acetate (60 mL). Next, the organic volatiles and water were removed with a rotary concentrator. After baking the result in a vacuum oven at 90° C. for 8 hours, Ionic compound (9) was obtained (with a structure of

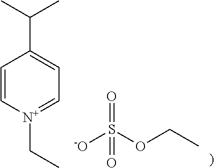

(white solid with a yield of 93%). The synthesis pathway of the above reaction was as follows:

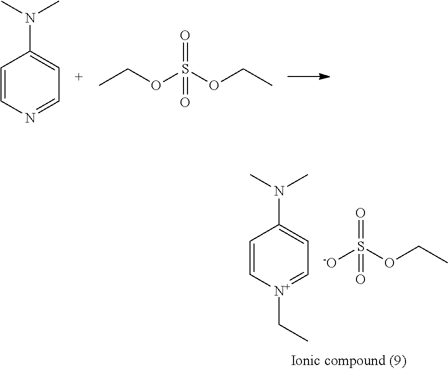

The measurement results of nuclear magnetic resonance spectrometry of Ionic compound (9) are shown below. ¹H NMR (400 MHz, ppm, CDCl₃): δ 8.26 (d, J=6.0 Hz, 2H), 6.98 (d, J=5.6 Hz, 2H), 4.31 (q, J=5.6 Hz, 2H), 4.15 (d, J=5.6 Hz, 2H), 3.25 (s, 6H), 1.54 (t, J=5.6 Hz, 3H), 1.31 (t, J=5.6 Hz, 3H).

### Preparation of Absorbent

Ionic compounds (1)-(9) were individually mixed with water. After stirring evenly, Absorbents (1)-(9) were obtained, wherein the weight ratio of the ionic compound (1) to water is 4:1. Chlorinated lithium was mixed with water. After stirring evenly, Absorbent (10) was obtained, wherein the weight ratio of chlorinated lithium to water was 1:2.

Ionic compound (10) (with a structure of

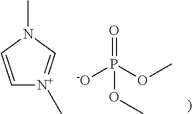

was provided. Ionic compound (10) was mixed with water, wherein the weight ratio of Ionic compound (10) to water was 4:1. After stirring evenly, Absorbent (11) was obtained.

Ionic compound (11) (with a structure of

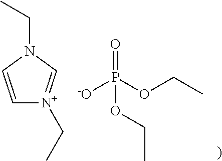

was provided. Ionic compound (11) was mixed with water, wherein the weight ratio of Ionic compound (11) to water was 4:1. After stirring evenly, Absorbent (12) was obtained.

Ionic compound (12) (with a structure of

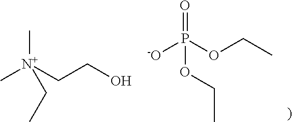

was provided. Ionic compound (12) was mixed with water, wherein the weight ratio of Ionic compound (12) to water was 4:1. After stirring evenly, Absorbent (13) was obtained.

Ionic compound (13) (with a structure of

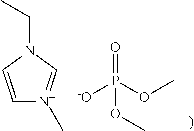

was provided. Ionic compound (13) was mixed with water, wherein the weight ratio of Ionic compound (13) to water was 4:1. After stirring evenly, Absorbent (14) was obtained.

### Evaluation of Dehumidification Capability

Absorbents (1)-(5) and (8)-(10) were individually disposed in an acrylic sealed box (internal volume: 20 cm×15.7 cm×3.9 cm), and then the relative humidity values within the acrylic sealed box was measured after 400 minutes. The results are shown in Table 1.

TABLE 1

| | relative humidity (%) |
|---|---|
| Absorbent (1) | 28 |
| Absorbent (2) | 42 |
| Absorbent (3) | 33 |
| Absorbent (4) | 33.5 |
| Absorbent (5) | 30 |
| Absorbent (8) | 48 |
| Absorbent (9) | 61 |
| Absorbent (10) | 40 |

In comparison with Ionic compound (2) of Example 2, Ionic compound (9) has the ethyl sulfate anionic moiety

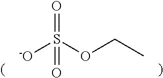

replaces with the diethyl phosphate anionic moiety

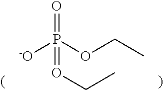

As shown in Table 1, in comparison with Absorbents (1)-(5), Absorbent (9) employing Ionic compound (9) exhibits relatively poor dehumidification capability. In addition, in comparison with Ionic compound (3) of Example 3, Ionic compound (8) has the N-methylpyridinium cationic moiety

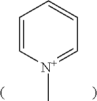

replaced with the 2-methyl-N-methylpyridinium cationic moiety

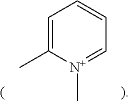

As shown in Table 1, in comparison with Absorbents (1)-(5), Absorbent (8) employing Ionic compound (8) exhibits relatively poor dehumidification capability.

### Measurement of Water Vapor Pressure

The water vapor pressure of Absorbents (1)-(5) were measured at 5° C., 10° C., 15° C., 20° C., 25° C., 30° C., 35° C., 40° C., 45° C., 50° C., 55° C., and 60° C., and the vapor pressure difference of Absorbents (1)-(5) at various temperature were determined, and the results are shown in Table 2. In addition, the water vapor pressure of Absorbents (6), (7), and (10)-(14 were measured at 5° C., 10° C., 15° C., 20° C., 25° C., 30° C., 35° C., 40° C., 45° C., 50° C., 55° C., and 60° C., and the vapor pressure difference of Absorbents (6), (7), and (10)-(14 at various temperature were determined, and the results are shown in Table 3.

The measurement method for the vapor pressure of the absorbent utilizes the melting-point model (reference: J. Chem. Eng. Data 2004, 49, 1550-1553) and includes following steps. Absorbent was disposed in a container, and the temperature was cooled to 5° C. After applying a vacuum until the evaporation of water vapor begins, the vacuum system was closed and the container was heated. The vapor pressure was measured using a pressure gauge (EJX310A, YOKOGAWA) at every 5° C.

TABLE 2

| | | Absorbent (1) | Absorbent (2) | Absorbent (3) | Absorbent (4) | Absorbent (5) |
|---|---|---|---|---|---|---|
| | | Ionic compound | | | | |
| | | Ionic | Ionic | Ionic | Ionic | Ionic |
| | | compound (1) | compound (2) | compound (3) | compound (4) | compound (5) |
| vapor | 5° C. | 0.09 | — | 0.1 | 0.15 | 0.14 |
| pressure | 10° C. | 0.23 | — | 0.21 | 0.26 | 0.25 |
| (kPa) | 15° C. | 0.4 | — | 0.35 | 0.41 | 0.38 |
| | 20° C. | 0.58 | — | 0.52 | 0.59 | 0.55 |
| | 25° C. | 0.85 | 1.05 | 0.75 | 0.83 | 0.77 |
| | 30° C. | 1.17 | 1.51 | 1.05 | 1.13 | 1.05 |
| | 35° C. | 1.59 | 2.07 | 1.43 | 1.51 | 1.4 |
| | 40° C. | 2.15 | 2.79 | 1.91 | 2 | 1.88 |
| | 45° C. | 2.86 | 3.7 | 2.49 | 2.65 | 2.49 |
| | 50° C. | 3.72 | 4.79 | 3.28 | 3.48 | 3.24 |
| | 55° C. | 4.76 | 6.17 | 4.24 | 4.51 | 4.22 |
| | 60° C. | 6.03 | 7.87 | 5.43 | 5.79 | 5.4 |

TABLE 3

| | | Absorbent | Absorbent | Absorbent | Absorbent | Absorbent | Absorbent |
|---|---|---|---|---|---|---|---|
| | | (6) | (7) | (10) | (12) | (13) | (14) |
| | | Ionic compound | | | | | |
| | | Ionic | Ionic | | Ionic | Ionic | Ionic |
| | | compound | compound | | compound | compound | compound |
| | | (6) | (7) | LiCl | (11) | (12) | (13) |
| vapor | 5° C. | 0.35 | 0.16 | 0.12 | 0.45 | 0.23 | 0.22 |
| pressure | 10° C. | 0.49 | 0.29 | 0.26 | 0.62 | 0.41 | 0.34 |
| (kPa) | 15° C. | 0.67 | 0.45 | 0.44 | 0.83 | 0.62 | 0.48 |
| | 20° C. | 0.9 | 0.64 | 0.67 | 1.09 | 0.85 | 0.66 |
| | 25° C. | 1.2 | 0.92 | 0.97 | 1.44 | 1.23 | 0.91 |
| | 30° C. | 1.58 | 1.26 | 1.38 | 1.87 | 1.61 | 1.21 |
| | 35° C. | 2.07 | 1.69 | 1.87 | 2.4 | 2.15 | 1.58 |
| | 40° C. | 2.69 | 2.27 | 2.52 | 3.11 | 2.89 | 2.08 |
| | 45° C. | 3.47 | 2.97 | 3.36 | 3.99 | 3.74 | 2.72 |
| | 50° C. | 4.46 | 3.85 | 4.36 | 5.03 | 4.83 | 3.54 |
| | 55° C. | 5.67 | 4.92 | 5.62 | 6.34 | 6.15 | 4.53 |
| | 60° C. | 7.22 | 6.22 | 7.18 | 8 | 7.98 | 5.78 |

The measured water vapor pressure of the Absorbent (10) of the disclosure (chlorinated lithium aqueous solution) is consistent with the values reported in the related literature (J. Chem. Eng. Data 1990, 35, 166-168; International Journal of Low-Carbon Technologies, Volume 11, Issue 4, 2016, 489-505).

The dehumidification capability of the absorbent can be evaluated via its water vapor pressure. When the absorbent is operating in an absorption device, it absorbs water vapor from the surrounding environment to reduce the humidity, driven by the difference between the water vapor pressure in the air and the saturated vapor pressure on the absorbent surface. The lowest relative humidity for dehumidifying air via the absorbent can be evaluated based on the water vapor pressure of the absorbent. A lower water vapor pressure of the absorbent indicates a stronger water-vapor absorption capacity of the absorbent (i.e., ionic compound aqueous solution) at the specific temperature.

As shown in Table 2, it was observed that the absorbent prepared from Ionic compounds (1) and (3)-(5) of the disclosure have a water vapor pressure less than or equal to 0.6 kPa at 5-20° C. As shown in Table 3, it was observed that the absorbent prepared from Ionic compounds (6), (7) and (11)-(13) of the disclosure have a water vapor pressure greater than or equal to 0.64 kPa at 5-20° C.

### Corrosion Test

Copper foils (C1100P) (with a size of 10×10×2 mm³) were provided and weighed. Next, Copper foils were disposed in sample vials with various absorbents (i.e. Absorbents (1), (3)-(8), and (10)-(13)) (8 mL) and tap water respectively. After stirring at 80° C. for 2 days, the copper foils was isolated, dried and weighed, and the percentage change in weight of copper foil was determined and the results are shown in Table 3. Next, the copper foils was replaced with aluminum foils (A5052) (with a size of 10×10×2 mm³) and stainless steel foils (SUS304) (with a size of 10×10×2 mm³) and the above steps were repeated. The results are shown in Table 4.

TABLE 4

| | percentage change in weight (%) | | |
|---|---|---|---|
| | stainless | aluminum | copper |
| | steel foil | foil | foil |
| Absorbent (1) | ±0.0000% | −0.04% | −0.06% |
| Absorbent (3) | ±0.0000% | 0.06% | −0.02% |
| Absorbent (4) | ±0.0000% | −0.04% | −0.01% |
| Absorbent (5) | ±0.0000% | ±0.0000% | −0.01% |
| Absorbent (6) | 0.03% | 0.14% | −0.49% |
| Absorbent (7) | 0.03% | ±0.0000% | −0.06% |
| Absorbent (8) | −0.01% | −0.09% | −0.10% |
| Absorbent (10) | −0.01% | −0.13% | −0.05% |
| Absorbent (11) | 0.04% | 0.20% | −0.20% |
| Absorbent (12) | −0.02% | 0.16% | −0.74% |
| Absorbent (13) | −0.01% | 0.10% | −0.12% |
| tap water | ±0.0000% | 0.08% | 0.03% |

As shown in Table 4, chlorinated lithium and Absorbents (6)-(8) and (11)-(13) exhibit various degrees of corrosiveness regarding to stainless steel foils, aluminum foils, and copper foils. Furthermore, according to Table 4, the absorbents prepared from the ionic compound of the disclosure (Absorbents (1)-(5)) demonstrate minimal corrosion regarding to stainless steel foils and aluminum foils, and also exhibit lower corrosion regarding to copper foils.

### Viscosity Measurement

The viscosities of Absorbents (1)-(4) at various temperatures were measured by a viscosity meter (DV-II+Pro, Brookfield), and the results are shown in Table 5.

TABLE 5

| | | Absor- | Absor- | Absor- | Absor- | Absor- |
|---|---|---|---|---|---|---|
| | | bent | bent | bent | bent | bent |
| | | (1) | (2) | (3) | (4) | (5) |
| viscosity | 15° C. | 119 | 85 | 84 | 58 | 52 |
| (cP) | 20° C. | 82 | 59 | 57 | 44 | 38 |
| | 25° C. | 53 | 44 | 39 | 32 | 26 |
| | 30° C. | 36 | 33 | 29 | 27 | 20 |
| | 35° C. | 28 | 25 | 21 | 20 | 16 |
| | 40° C. | 21 | 19 | 16 | 16 | 12 |
| | 45° C. | 18 | 17 | 12 | 13 | 10 |
| | 50° C. | 15 | 15 | 11 | 12 | 9 |

Viscosity is also an important parameter for evaluating absorbents. When the viscosity of an absorbent is too low, it becomes prone to liquid carryover, where liquid is entrained and carried out with the air, resulting in environmental pollution. On the other hand, when the viscosity of an absorbent is too high, it reduces the flowability of the absorbent, increasing the energy consumption of the pump. As shown in Table 5, the absorbents prepared from the ionic compounds of the disclosure, possess the viscosity of 50 cP to 120 cP at 15° C., suitable for applications in dehumidification systems that adopt spray or liquid flow processes.

### Odor and Degradation Evaluation

As the air introduced by the dehumidification system comes into direct contact with the absorbent, if an ionic compound produces odors, it may be less suitable for use in air condition systems. Absorbents (1)-(5) prepared from the ionic compound of the disclosure were stored at room temperature and atmospheric pressure for 180 days. Following the evaluation, Absorbents (1)-(5) did not undergo degradation and did not exhibit any noticeable odor generation.

Accordingly, the absorbent including the ionic compound of the disclosure exhibits a higher water-vapor absorption capacity and a lower desorption temperature thereby increasing the dehumidification efficiency of the absorption device that utilizes the absorbent. Additionally, the liquid absorbent containing the ionic compound of the disclosure offers advantages such as odorless, antibacterial, low corrosiveness, fluidity, and excellent room temperature stability, which can be widely applied to various dehumidification devices.

It will be clear that various modifications and variations can be made to the disclosed methods and materials. It is intended that the specification and examples be considered as exemplary only, with the true scope of the disclosure being indicated by the following claims and their equivalents.

What is claimed is:

1. An ionic compound, having a structure represented by Formula (I):
AB Formula (I)
wherein, A is 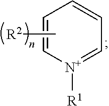
B is 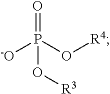
R¹ is C1-C6 alkyl group; R² is independently hydroxy group, amino group, C1-C6 alkyl group, C1-C6 alkoxy group, C1-C12 alkylamino group, C2-C7 alkoxycarbonyl group, C2-C7 alkanoyloxy group, carbamoyl group, C2-C13 alkylcarbamoyl group, or C2-C7 alkanoylamino group; n is 1, 2, 3, 4, or 5; and R³ and R⁴ are independently H, or C1-C6 alkyl group.

2. The ionic compound as claimed in claim 1, wherein A is 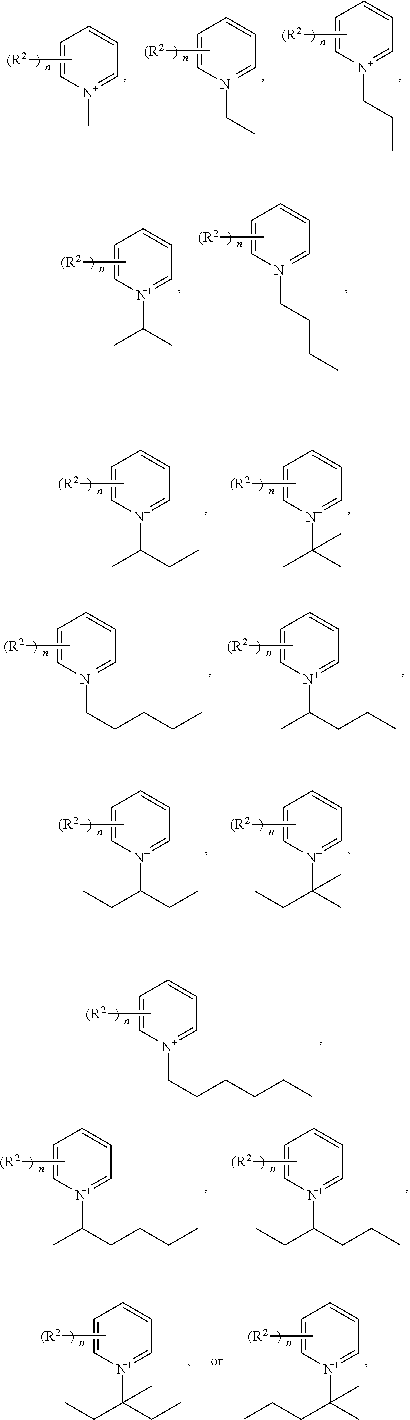
wherein R² is independently hydroxy group, amino group, C1-C6 alkyl group, C1-C6 alkoxy group, C1-C12 alkylamino group, C2-C7 alkoxycarbonyl group, C2-C7 alkanoyloxy group, carbamoyl group, C2-C13 alkylcarbamoyl group, or C2-C7 alkanoylamino group; n is 1, 2, 3, 4, or 5; and R³ and R⁴ are independently H or C1-C6 alkyl group.

3. The ionic compound as claimed in claim 1, wherein A is 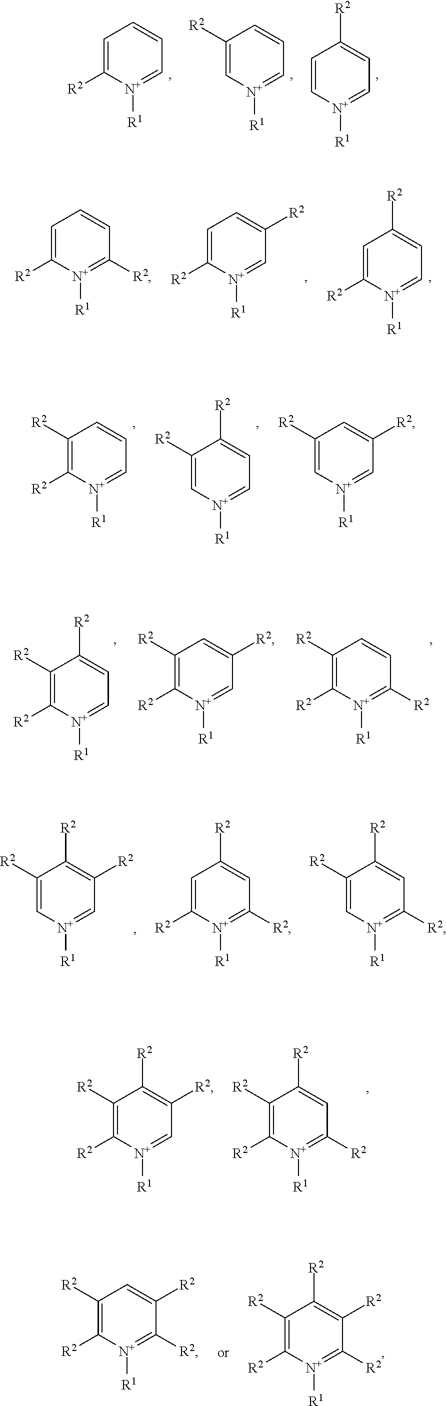 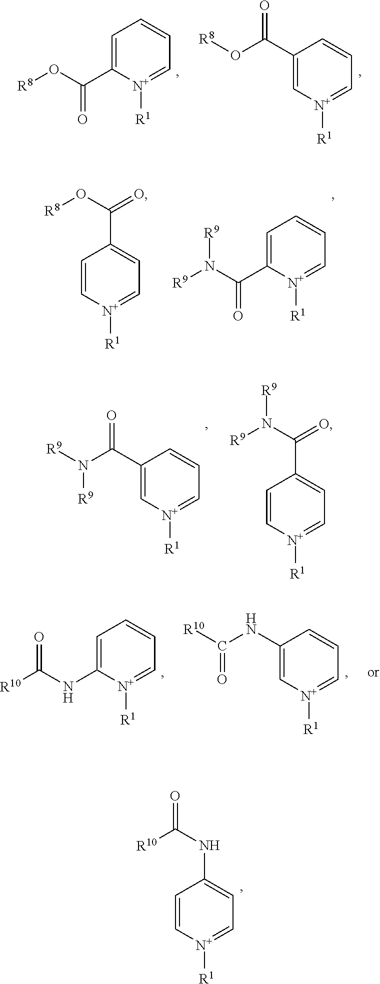
wherein R¹ is C1-C6 alkyl group; R² is independently hydroxy group, amino group, C1-C6 alkyl group, C1-C6 alkoxy group, C1-C12 alkylamino group, C2-C7 alkoxycarbonyl group, C2-C7 alkanoyloxy group, carbamoyl group, C2-C13 alkylcarbamoyl group, or C2-C7 alkanoylamino group.

4. The ionic compound as claimed in claim 1, wherein A is 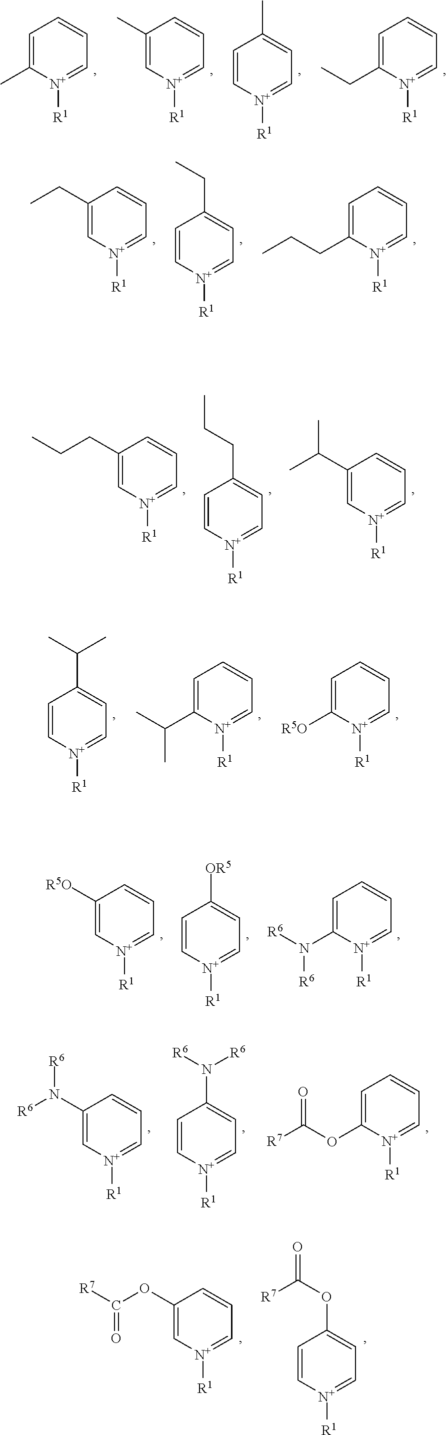 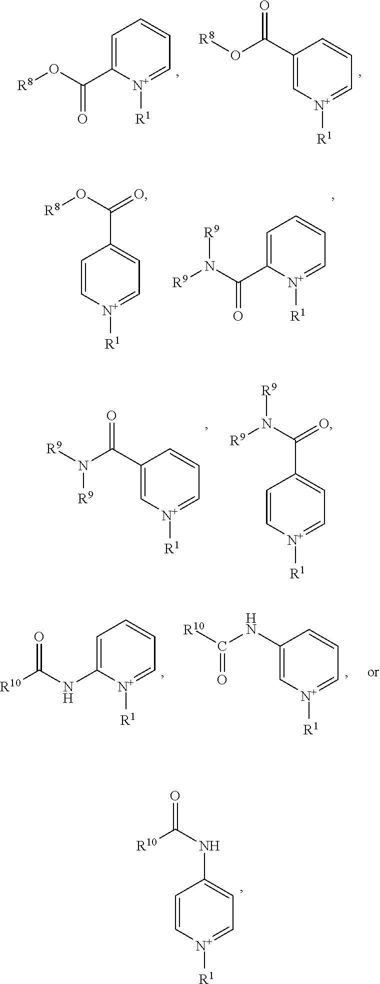
wherein R¹, R⁷, R⁸, and R¹⁰ are independently C1-C6 alkyl group; and R⁵, R⁶ and R⁹ are independently H, or C1-C6 alkyl group.

5. The ionic compound as claimed in claim 1, wherein B is

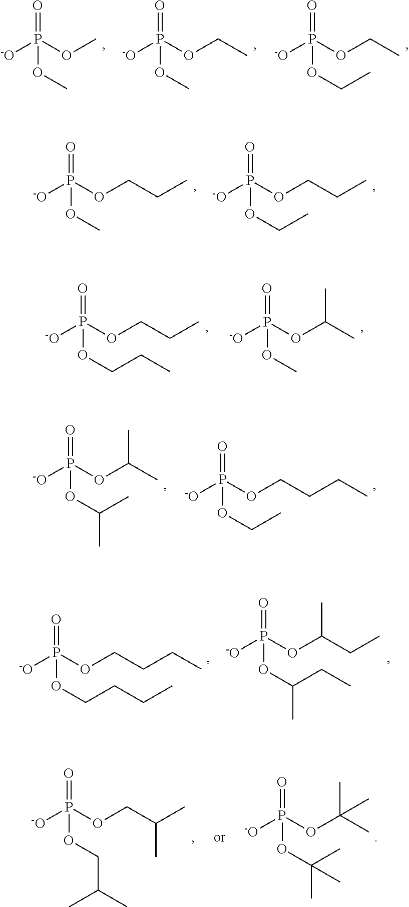

6. An absorbent, comprising:
an ionic compound, wherein the ionic compound is the ionic compound as claimed in claim 1.

7. The absorbent as claimed in claim 6, further comprising:
a solvent, wherein the weight ratio of the ionic compound to the solvent is 1:9 to 9:1.

8. The absorbent as claimed in claim 7, wherein the solvent is water, methanol, ethanol, propanol, butanol, ammonia, or a combination thereof.

9. The absorbent as claimed in claim 7, wherein the solvent is water, and the weight ratio of ionic compound to water is 9:1 to 4:1, wherein the absorbent has a viscosity of 50 cP to 120 cP at 15° C.

10. An absorption device, comprising:
a chamber; and
an absorbent disposed in the chamber, wherein the absorbent is the absorbent as claimed in claim 6.
